(19) United States
(12) Patent Application Publication
(10) Pub. No.: US 2015/0177223 A1
(43) Pub. Date: Jun. 25, 2015

(54) System and Method for Detecting and Diagnosing Schizophrenia and Depression

(71) Applicant: Yale University, New Haven, CT (US)

(72) Inventor: Handan Gunduz-Bruce, Woodbridge, CT (US)

(21) Appl. No.: 14/416,842

(22) PCT Filed: Jul. 23, 2013
(86) PCT No.: PCT/US13/51643

§ 371 Date: Jan. 23, 2015

Related U.S. Application Data
(60) Provisional application No. 61/674,666, filed on Jul. 23, 2012.

(51) Int. Cl. G01N 33/49; G01N 33/74

(52) U.S. Cl. CPC G01N 33/49; G01N 33/74; G01N 2800/302; G01N 2800/304

(57) ABSTRACT

The invention relates to methods that can detect the abnormal function of the N-methyl-D-aspartate (NMDA) receptor, which can ascertain whether an individual has vulnerability for schizophrenia or depression. The invention relates to measuring blood plasma osmolality and the concentration of least one biomarker in conjunction with hypertonic solution infusion as a method for detecting or diagnosing schizophrenia or depression. The invention further relates to using arginine-vasopressin (AVP), neurophysin II, and copeptin as biomarkers in a method for detecting or diagnosing schizophrenia or depression.

### CROSS-REFERENCE TO RELATED APPLICATIONS

The present application claims priority under 35 U.S.C. 119(e) to U.S. Provisional Patent Application No. 61/674,666, filed Jul. 23, 2012 which is herein incorporated by reference in its entirety.

### BACKGROUND OF THE INVENTION

Schizophrenia is a serious mental illness with a 12-month prevalence of 1% of the United States adult population. Major depression is more prevalent at 7%. Although schizophrenia has been recognized since the beginning of medical history, it remains a tremendous burden to individuals and society. The World Health Organization's (WHO) 2002 report ranked schizophrenia as the 3rd largest burden among all neuropsychiatric disorders. The per capita resources spent on care for schizophrenia in the U.S. is estimated at $2.3 million. (Blomqvist et al., 2006, J. Ment. Health Policy Econ. 9(4):177-183). Depression is also a costly disorder. The total economic burden of depression in the U.S. was estimated to be $83.1 billion in 2000.

Currently, there are no biological or physiological mechanisms for detecting and diagnosing early stage psychiatric illnesses such as schizophrenia, nor are there mechanisms for distinguishing early stage schizophrenia from depression. Therefore, the diagnoses for neuropsychiatric disorders such as schizophrenia and depression rely on subjective reporting and observation of behavior. As a result, there is a significant amount of diagnostic uncertainty, particularly in the early stages of psychiatric illnesses. Specifically, early schizophrenia, i.e., prodromal stage, resembles depression and is difficult to accurately diagnose and treat. Studies show that only 35% of subjects with prodrome symptoms develop psychosis in 2½ years of follow-up (Cannon et al., 2008, Arch Gen Psychiatry, January; 65(1):28-37). This raises significant ethical concerns about the treatment of subjects during this early stage. At present, clinical practice runs the risk of exposing individuals with uncertain diagnosis to antipsychotic medications, when they may not be destined to develop schizophrenia, but instead may have only a mood disorder, such as depression. Conversely, during this early stage of diagnostic uncertainty, delaying antipsychotic treatment for individuals who are destined to develop schizophrenia is also a major problem. It is also notable that in some prodromal subjects, no psychiatric disorders develop, and these symptoms appear to be part of normal adolescence.

Thus, there is a need in the art for biological or physiological mechanism, such as a biomarker for neuropsychiatric disorders such as schizophrenia and depression that could be used as an objective diagnostic tool in the detecting and diagnosing of psychiatric illnesses, to evaluate treatment response, and to eventually develop superior medications than what are currently available. The present invention addresses this unmet need in the art.

### SUMMARY OF THE INVENTION

The present invention relates to a method of diagnosing schizophrenia or depression in a subject. In one embodiment, the method of the present invention comprises the steps of: administering a hypertonic solution to a subject; measuring blood plasma osmolality in the subject; measuring the concentration of at least one biomarker in the subject; determining the value of a diagnostic parameter for the subject using the blood plasma osmolality in the subject and the concentration of at least one biomarker in the subject; and diagnosing the subject with schizophrenia when the value of the diagnostic parameter for the subject is less than a reference value of the diagnostic parameter. In another embodiment, the method of the present invention comprises the steps of: administering a hypertonic solution to a subject; measuring blood plasma osmolality in the subject; measuring the concentration of at least one biomarker in the subject; determining the value of a diagnostic parameter for the subject using the blood plasma osmolality in the subject and the concentration of at least one biomarker in the subject; and diagnosing the subject with depression when the value of the diagnostic parameter for the subject is greater than a reference value of the diagnostic parameter.

In various embodiments, the diagnostic parameter of the methods of the present invention is based on the slope and/or threshold values derived from the regression line of a plot of biomarker concentration versus blood plasma osmolality. In one embodiment, the diagnostic parameter is a Disease Vulnerability Index (DVI), based on a calculation of the slope and threshold values derived from the regression line of a plot of biomarker concentration versus blood plasma osmolality. In another embodiment, the diagnostic parameter is the value of the slope of the regression line of a plot of biomarker concentration versus blood plasma osmolality. In yet another embodiment, the diagnostic parameter is the threshold value of a plot of biomarker concentration versus blood plasma osmolality.

In various embodiments, the reference value for the diagnostic parameter of the methods of the present invention is based on measurements of the blood plasma osmolality and the concentration of at least one biomarker in one or more healthy control subjects, one or more subjects with schizophrenia, and/or one or more subjects with depression. In one embodiment, reference value for the slope of the regression line of a plot of biomarker concentration versus blood plasma osmolality for diagnosing schizophrenia is about 0.060. In one embodiment, reference value for the threshold of the regression line of a plot of biomarker concentration versus blood plasma osmolality for diagnosing schizophrenia is about 260. In one embodiment, reference value for the slope of the regression line of a plot of biomarker concentration versus blood plasma osmolality for diagnosing depression is about 0.130.

In one embodiment, the biomarker used in the methods of the present invention is arginine-vasopressin. In another embodiment, the biomarker is neurophysin II. In yet another embodiment, the biomarker is copeptin.

The present invention also relates to a kit for detecting or diagnosing schizophrenia or depression, comprising at least one biomarker assay, a hypertonic solution for infusion, and instructional material. In one embodiment, the biomarker assay is for measuring arginine-vasopressin concentration. In another embodiment, the biomarker assay is for measuring copeptin concentration. In yet another embodiment, the biomarker assay is for measuring neurophysin II concentration. In one embodiment, the kit further comprises an assay for measuring blood plasma osmolality. In one embodiment, the assay is based on a monoclonal antibody.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a graph depicting the linear significant increase of plasma osmolality over time in healthy control subjects, subjects with schizophrenia and depression. The magnitude of the increase among the groups was not statistically different than each other.

FIG. 2 is a set of graphs depicting a plot of the concentration of arginine-vasopressin (P_{[AVP]}) versus plasma osmolality (P_{Osm}), referred as “P_{[AVP]} Response to P_{Osm}” in blood samples during hypertonic saline infusion (HSI) in representative subjects from three groups, healthy control subjects, schizophrenic subjects, and depressed subjects.

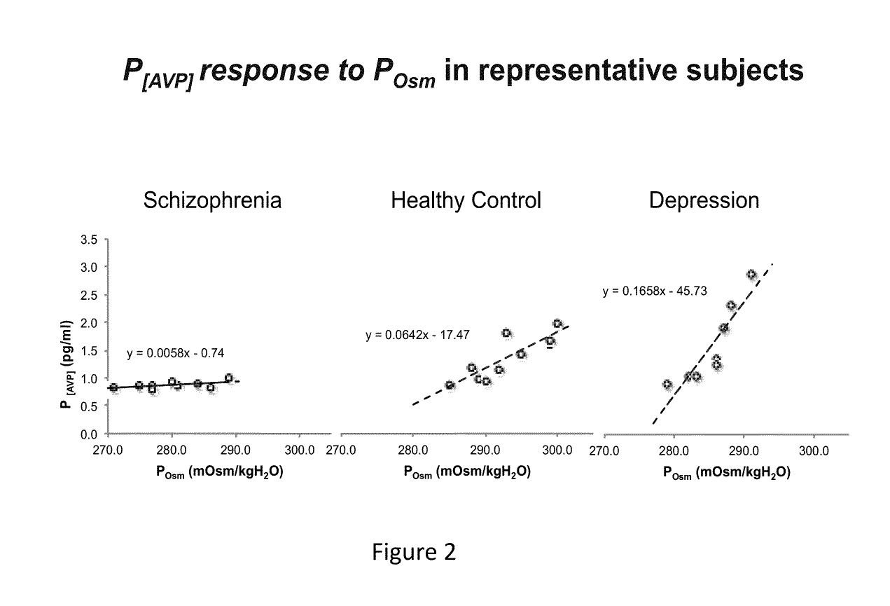

FIG. 3, comprising FIGS. 3A and 3B, is a set of graphs depicting the slope (FIG. 3 A) and threshold values (FIG. 3 B) of the relationship between P_{[AVP]} and P_{Osm} in all subjects of the three groups, healthy control subjects, schizophrenic subjects and depressed subjects.

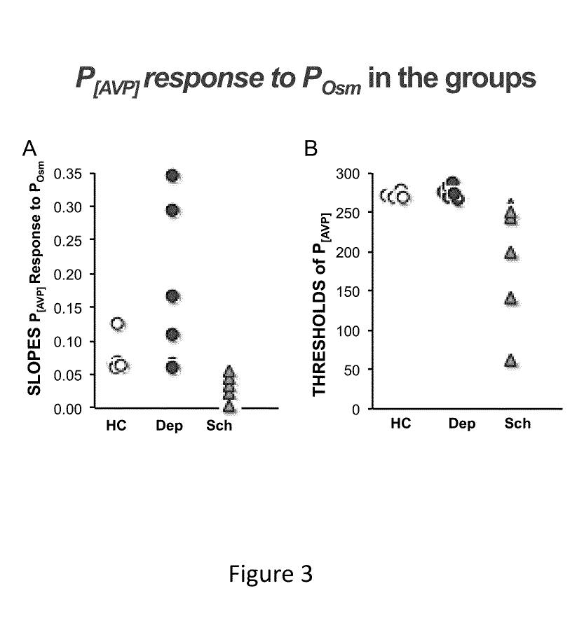

FIG. 4 is a graph showing combined data of slope and threshold values in all three groups.

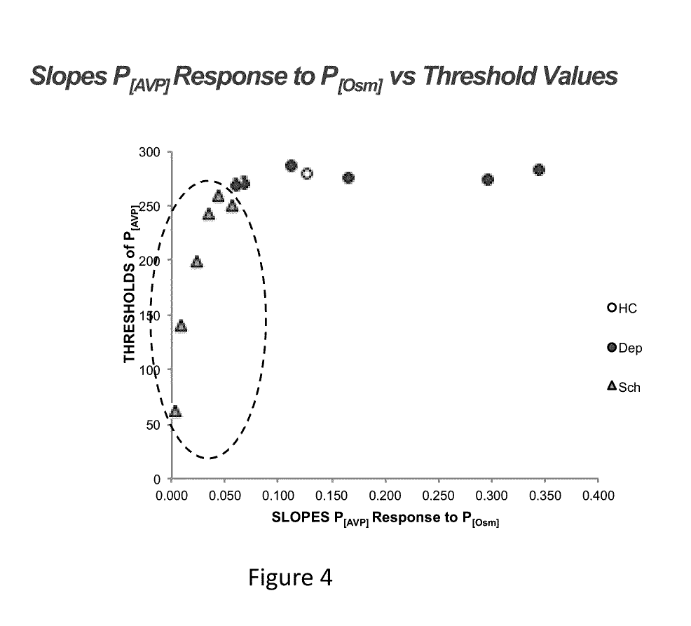

### DETAILED DESCRIPTION

It is to be understood that the figures and descriptions of the present invention have been simplified to illustrate elements that are relevant for a clear understanding of the present invention, while eliminating, for the purpose of clarity, many other elements found in the fields of neuropsychiatric disorders and biomarkers for neuropsychiatric disorders. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal, including a human, wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the frequency or severity of a sign or symptom of a disease or disorder, experienced by a subject is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The phrase “biological sample” as used herein, is intended to include any sample comprising a cell, a tissue, or a bodily fluid, such as blood. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

The term “biomarker” refers to a substance or indicator that is directly or indirectly used for diagnosing the presence or absence of a disease, susceptibility or vulnerability to a disease, the degree of the disease, the possibility for alleviation of the disease, or the degree of alleviation, or for screening substances which might be useful for preventing or treating a disease. As contemplated herein, the cleavage products of prepro-vasopressin, namely arginine-vasopressin, neurophysin II and copeptin, are each examples of a biomarker.

As used herein, an “instructional material” includes a publication, a recording, a diagram, or any other medium of expression which can be used to communicate the use of a method, composition, assay, or delivery system of a kit that can be used for detecting or diagnosing the various diseases or disorders recited herein. The instructional material of the kit can, for example, be affixed to a container which contains the identified method, composition, assay, or delivery system of the invention or be shipped together with a container which contains the identified method, compound, composition, assay, or delivery system. Alternatively, the instructional material can be shipped separately from the container with the intention that the instructional material and the method, composition, assay, or delivery system be used cooperatively by the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention relates to evidence implicating the brain glutamate system, in particular the diminished function of the N-methyl-D-aspartate (NMDA) receptor, an ionotropic glutamate receptor system, in the pathophysiology of schizophrenia. In addition, the present invention relates to the determination that enhanced function of the same receptor system indicates depression. The lack of suitable ligands for this receptor system has hampered the development of glutamate biomarkers for these disorders prior to the discoveries on which the present invention is based. The methods of the present invention can be used to differentiate subjects with schizophrenia from subjects with major depression and from healthy subjects, based in part on a biological measure.

Accordingly, the present invention relates to methods for detecting abnormalities in the NMDA receptor system in subjects with schizophrenia or depression by using an osmotic probe and stimulating hyperosmolality-induced release of arginine-vasopressin (AVP). The methods of the present invention can be used to ascertain whether an individual has vulnerability for schizophrenia or depression. In one embodiment, the invention relates to the measurement and/or detection of arginine-vasopressin as a biomarker for schizophrenia and depression. In another embodiment, the invention relates to the measurement and/or detection of copeptin and neurophysin II, which are co-secreted with arginine-vasopressin, as a biomarker for schizophrenia and depression.

Arginine-vasopressin (AVP), also known as vasopressin, argipressin, or antidiuretic hormone (ADH), is a hormone and neuropeptide stored in the posterior pituitary that plays important roles in physiological functions, such as renal free water clearance and blood pressure regulation, and psychological functions, such as social attachment. Subjects with schizophrenia have deficits in these physiological and psychological areas. Arginine-vasopressin release is critically regulated by the NMDA receptor system.

Early physiological studies in schizophrenia, prior to the availability of antipsychotic treatments, found that some patients demonstrate polydipsia and increased urinary output, i.e., excessive water intake, which may lead to hyponatremia. Although plasma AVP concentration (P_{[AVP]}) is highly sensitive to changes in plasma osmolality (P_{Osm}), previous hypertonic saline infusion (HSI) studies documented lower osmotic thresholds and lower slopes for the P_{Osm} vs. P_{[AVP]} relationship in the polydipsic schizophrenics as a group. Other studies have found alterations in threshold and sensitivity of P_{[AVP]} by examining the correlation between plasma osmolality and AVP in patients without polydipsia/hyponatremia. Moreover, these physiological alterations seem to correlate with illness severity, with polydipsia/hyponatremia being associated with worse outcomes and poor response to treatment. Because the release of AVP is mediated by NMDA receptors, these findings are also consistent with the implicated NMDA receptor hypofunction in schizophrenia.

Abnormalities in water regulation in schizophrenia have been clearly documented prior to the availability of antipsychotics, and observed in antipsychotic-free patients, indicating that these abnormalities are not caused by exposure to antipsychotic medications. Specifically, some patients with schizophrenia drink an excessive amount of water, a symptom known as polydipsia, and have increased urinary output. Some schizophrenic patients also develop hyponatremia. AVP is released from the posterior pituitary and is highly sensitive to changes in plasma osmolality (P_{Osm}), for example hyperosmolality is a potent stimulator of AVP secretion. However, plasma AVP concentrations (P_{[AVP]}) are lower in schizophrenic subjects than in non-schizophrenic subjects for given P_{Osm} values, and AVP secretion occurs at lower thresholds of P_{Osm}. In addition, hypertonic saline infusion studies have found lower slopes of P_{[AVP]} versus P_{Osm} in schizophrenic subjects, in other words lower sensitivity independent of polydipsia/hyponatremia.

Importantly, water regulation abnormalities manifest themselves as a function of the illness severity. Patients with polydipsic hyponatremia have more severe illness with higher symptom scores, are more dysfunctional, and respond more poorly to treatment, suggesting that water regulation abnormalities may signify a continuum, rather than a categorical representation in this illness.

Glutamatergic synapses of osmosensitive cells that terminate on magnocellular neurosecretory cells (MNCs) contain NMDA receptors. Magnocellular neurosecretory cells store and secrete AVP. NMDA agonists increase the release of AVP and NMDA antagonists reduce the release of AVP. Therefore, the reduction of AVP response to HSI in schizophrenia is consistent with the widely implicated NMDA receptor hypofunction in its pathophysiology.

Previous biomarker studies in schizophrenia have focused on genomics, serum and cerebrospinal fluid (CSF) based proteomics and metabolomics approaches, but these studies have found nonspecific changes and have not been well replicated. (Lakhan et al., 2009 Behav. Brain Funct. 5:2; Vasic et al., Eur. Arch. Psychiatry Clin. Neurosci. December 2011; Kluge et al., 2011 Expert Rev. Mol. Diagn. 11(7):721-733). Imaging studies do not provide the desired level of sensitivity and specificity for biomarker development in schizophrenia despite continuing efforts. (Carter et al., 2012 Schizophr. Bull. 38(1):26-33). Therefore, the findings and methods described herein represent a significant departure from other biomarker studies in the field in that they are built upon a clear physiological dysfunction.

Several prior studies examined the water regulation abnormalities in schizophrenia, but focused on categories of patients showing overt symptoms of polydipsia/hyponatremia, not recognizing the abnormalities as a continuum. (Kishimoto et al., 1989 Jpn. J. Psychiatry Neurol. 43(2):161-169; Goldman et al., 1996 J. Clin. Endocrinol. Metab. 81(4): 1465-1470; Goldman et al., 1988 N. Engl. J. Med. 318(7):397-403). This is an important distinction because hypertonic saline infusion studies and other studies have revealed that water regulation abnormalities are also present in patients without symptoms of polydipsia/hyponatremia, showing decreased AVP response, i.e. low sensitivity, and lower thresholds. (Goldman et al., 1996 J. Clin. Endocrinol. Metab. 81(4): 1465-1470; Goldman et al., 1988 N. Engl. J. Med. 318(7):397-403; Sarai et al. 1989 Psychiatry 26(6): 576-580; Hundt et al., 2001 World J. Biol. Psychiatry 2(1):27-33). Moreover, water regulation abnormalities manifest themselves as a function of the illness severity: polydipsic/hyponatremic patients have more severe illness with higher symptom scores, are more dysfunctional, and are poorer responders to treatment. (Sleeper, 1935 Am J. Psychiatry 91:1019-1031; Kirch et al., 1985 J. Clin. Psychiatry 46(5):179-181; Poirier et al., 2010 Schizophr. Res. 118(1-3): 285-292). Based on these findings, the water regulation abnormalities in schizophrenia are treated as a continuous variable in the disclosure herein.

Critical criteria for biomarker development are association with illness, heritability, state-independence, i.e., manifesting regardless of illness episodes, and reproducibility. As yet, there are no biomarkers for schizophrenia that meet all these criteria. Previous physiological studies have shown that the AVP response to hypertonic saline is highly heritable and reproducible. (Zerbe et al., 1991 J. Lab. Clin. Med. 117(1):51-59). Therefore, based on the findings discussed herein, one skilled in the art could use AVP dysregulation as a method for detecting or diagnosing schizophrenia.

The NMDA receptor complex has also been implicated in the pathophysiology of depression (Cannon et al., 2008, Arch Gen Psychiatry, January; 65(1):28-37; Sanacora et al., 2008, Nat Rev Drug Discov., 7(5):426-37; Machado-Vieira et al., 2009, Neuroscientist, 15(5):525-39). Several studies using ¹H-MRS have shown changes in glutamate concentration compared to healthy controls, and reduced number and density of glial cells. In contrast to the observations in schizophrenia, clinical trials in depressed subjects revealed that treatment with NMDA antagonists like ketamine produces significant improvement (Berman et al., 2000, Biol Psychiatry, 47(4):351-4; Zarate et al., 2006, Arch Gen Psychiatry, August; 63(8):856-64; Valentine et al., 2011, Psychiatry Res., February 28; 191(2):122-7; Machado-Vieira et al., 2012, Pharmacol Biochem Behav., 100(4):678-87), suggesting an overactive NMDA receptor function in depression.

A previous study found that patients diagnosed with major depressive disorder have higher P_{[AVP]} than healthy controls (van Londen et al., 1997, Neuropsychopharmacology, October; 17(4):284-92). An overactive NMDA receptor function in depression will result in an increased AVP response to hypertonic saline (i.e., higher P_{[AVP]} response to P_{Osm}) compared to healthy controls. Because of an hypofunction of NMDA receptor in schizophrenia, subjects with depression and schizophrenia will be further apart from each other based on the diagnostic test of the present invention, i.e., higher than normal P_{[AVP]} response to P_{Osm} in depression and lower than normal P_{[AVP]} response to P_{Osm} in schizophrenia.

As demonstrated herein, the slope of the regression line of P_{[AVP]} vs P_{Osm} (P_{[AVP]} response to P_{Osm}) during HSI is sufficient to distinguish healthy control subjects and depressed subjects from schizophrenics. Using Receiver Operating Characteristic (ROC) curves, the Area Under the Curve (AUC) can be calculated. From this AUC calculation, a DVI cutoff can be selected that corresponds to the desired sensitivity and specificity in distinguishing schizophrenic subjects from healthy subjects or depressed subjects. For example, a value of less than 0.060 for the slope of the regression line of P_{[AVP]} vs P_{Osm} can distinguish schizophrenic subjects from both depressed and healthy control subjects, where all of the schizophrenic subjects had a value of less than 0.060. From this, a DVI function can be calculated that incorporates both the slope and the intercept, i.e., threshold for AVP, value of the regression line, which will result in a higher sensitivity and specificity of the methods described herein.

The method described herein can also help determine subjects with depression with extreme slope values, representing potentially a subgroup of depressed subjects with very high NMDA receptor function. For example, based on the results of experiments described herein, a slope value of 0.130 detects this extreme subgroup of subjects with depression. These individuals with extremely high slope values can be candidates for glutamatergic antidepressants that are currently under development.

Accordingly, the present invention relates to methods for determining whether an individual has vulnerability towards schizophrenia or depression. In various embodiments, the invention can be used to differentiate patients with schizophrenia from patients with depression or healthy patients based on a biological measure.

In one embodiment, the invention contemplates that measuring the osmotic regulation of AVP can be used to determine if a subject has vulnerability for schizophrenia. In one embodiment, the invention contemplates that measuring the osmotic regulation of AVP can be used to determine if a subject has vulnerability for depression. In one embodiment, the invention relates to a method for detecting and diagnosing schizophrenia based on AVP dysregulation. In one embodiment, the invention relates to a method for detecting and diagnosing depression based on AVP dysregulation.

In another embodiment, the method may include measuring AVP concentration after hypertonic saline infusion or other means to increase plasma osmolality, such as concentrated mannitol infusion, in the detection and/or diagnosis of schizophrenia. In another embodiment, the method may include measuring AVP concentration after hypertonic saline infusion or other means to increase plasma osmolality, such as concentrated mannitol infusion, in the detection and/or diagnosis of depression. Such a method is based on the findings herein described that when schizophrenic subjects receive hypertonic saline infusion they do not release the required level of AVP into the bloodstream because of the NMDA receptor hypofunction, resulting in lower slopes for osmotic release of AVP. Conversely, such a method is based on the findings, described herein, that when depressed subjects receive hypertonic saline infusion they release more than the required level of AVP into the bloodstream because of the NMDA receptor overfunction, resulting in higher slopes for osmotic release of AVP.

In one embodiment of the present invention, copeptin is used as a biomarker in a method for the detection or diagnosis of schizophrenia or depression. In another embodiment, neurophysin II is used as a biomarker in a method for the detection or diagnosis of schizophrenia or depression. Copeptin, the C-terminal moiety of prepro-vasopressin, and neurophysin II are co-secreted with arginine-vasopressin (AVP). Plasma AVP and copeptin correlate strongly over a wide range of osmolalities in healthy individuals. Therefore, the measurement of copeptin, which remains stable for several days, is a useful alternative to measuring AVP concentration. (Balanescu et al., J Clin Endocrinol Metab 96: 1046-1052, 2011). Based on the disclosure discussed herein, a skilled artisan could use copeptin and/or neurophysin II measurements instead of, or in conjunction with, AVP measurements in order to diagnose schizophrenia and depression. However, the present invention is not limited to any particular method of administration or treatment regimen.

In one embodiment, the invention relates to a method for detecting and diagnosing schizophrenia based on the findings, described herein, that subjects with schizophrenia have lower slopes and/or thresholds for the relationship between P_{[AVP]} and P_{Osm} during hypertonic saline infusion than healthy or depressed control subjects. Conversely, in one embodiment, the invention relates to a method for detecting and diagnosing depression based on the findings, described herein, that subjects with depression have higher slopes and/or thresholds for the relationship between P_{[AVP]} and P_{Osm} during hypertonic saline infusion than most healthy and all schizophrenic subjects.

In one embodiment, the invention relates to a method for detecting and diagnosing schizophrenia and depression in which a hypertonic solution, such as hypertonic saline solution, is continually infused into a human subject while periodic samples are taken from the subject before, during, or after the hypertonic solution infusion. The method may include measuring the plasma osmolality and the plasma concentration of AVP in the samples. As described herein, healthy control subjects show a narrow range with a linear relationship between plasma osmolality and AVP concentration, while schizophrenic subjects show a distinctly blunted response, and the depressed subjects demonstrate a high range of variability including a subgroup with extreme responses. Further, the methods of the present invention may include distinguishing schizophrenic subjects from both depressed and healthy subjects based on the measurements of plasma osmolality and the plasma concentration of AVP, copeptin, neurophysin II or other biomarker. Similarly, the methods of the present invention may include distinguishing a subgroup of depressed subjects from both schizophrenic and healthy subjects based on the measurements of plasma osmolality and the plasma concentration of AVP, copeptin, neurophysin II or other biomarker.

In one embodiment, the invention relates to the use of a novel AVP assay in conjunction with hypertonic solution infusion that standardizes the procedure herein described for wide-spread use. In one embodiment, the assay is a monoclonal antibody-based assay. In another embodiment, the assay is a polyclonal antibody-based assay. In various embodiments, the invention can be used to clarify diagnosis when there is diagnostic uncertainty in young patients with prodromal symptoms of psychiatric illness, and also in adult patients.

In one embodiment, the invention relates to a method in which a hypertonic solution is administered to a subject for a period of time, for example at least 30 min, but preferably 2 hours, for the purposes of determining if the subject has vulnerability for schizophrenia or depression. After the hypertonic saline infusion, the subject waits for a period of time as a stabilization period, for example at least 5 minutes, but preferably 30 minutes. After the stabilization period, the subject drinks plain water for a period of time, for example 10-20 ml/kg of water for at least 5 minutes, but preferably 30 minutes. Following water ingestion, the subjects rest for period of time, for example at least 5 minutes, but preferably 60 minutes. However, the values for the time periods and water amounts are not limited to the values listed herein, and can be any value as would be understood by a person with ordinary skill in the art.

After the water ingestion and rest period, blood plasma osmolality (P_{Osm}) and the concentration of at least one biomarker, for example AVP (P_{[AVP]}), are then determined through measurements of a series of blood samples from the subject. The P_{Osm} and P_{[AVP]} measurements are used to determine a diagnostic parameter of the subject. Once the value of the diagnostic parameter for the subject with an uncertain diagnosis is obtained, this value is compared to a cutoff or reference value for the diagnostic parameter. The reference value for the diagnostic parameter is based on P_{Osm} and P_{[AVP]} measurements from healthy control subjects, and/or subjects previously diagnosed with unipolar depression, and schizophrenia. By comparing the value of the diagnostic parameter of the subject with an uncertain diagnosis with values of the diagnostic parameters representative of healthy control subjects, subjects with unipolar depression, and schizophrenia, i.e., reference parameters, an evaluation can be made as to which of the three groups the diagnostic parameter of the subject with uncertain diagnosis is consistent with.

Accordingly, the methods of the present invention relate to the determination of a diagnostic parameter in a subject, wherein the diagnostic parameter is based on measurements of blood plasma osmolality and the concentration of at least one biomarker in the subject. In one embodiment, the diagnostic parameter is a slope value derived from the regression line of a plot of biomarker concentration versus blood plasma osmolality. In another embodiment, the diagnostic parameter is a threshold value derived from the regression line of a plot of biomarker concentration versus blood plasma osmolality. In another embodiment, the diagnostic parameter is a Disease Vulnerability Index (DVI) based on both the slope and threshold values derived from the regression line of a plot of biomarker concentration versus blood plasma osmolality. However, the diagnostic parameter is not limited to the parameters described herein, and can be any parameter that is based on biomarker concentration and blood plasma osmolality measurements, and that can be used to distinguish healthy subjects from subjects with schizophrenia or depression.

The value of the diagnostic parameter of the present invention can be compared to a cutoff, or reference value, of the diagnostic parameter to determine if the subject has schizophrenia or depression, or if the subject is healthy. In one embodiment, if the value of the diagnostic parameter for the subject is less than the value of the cutoff or reference value for the diagnostic parameter, then the subject can be diagnosed with schizophrenia. In one embodiment, if the value of the diagnostic parameter for the subject is more than the value of the cutoff or reference value for the diagnostic parameter, then the subject can be diagnosed with depression.

In one embodiment, the cutoff or reference value used in the method of the present invention can be determined by measuring blood plasma osmolality and the concentration of at least one biomarker in one or more control subjects. In another embodiment, the cutoff or reference value used in the method of the present invention can be obtained from previous measurements of blood plasma osmolality and the concentration of at least one biomarker in one or more control subjects. For example, the cutoff or reference value for the diagnostic parameter can be determined based on statistical analysis of significant numbers of measurements of blood plasma osmolality and the concentration of at least one biomarker in healthy subjects, subjects with schizophrenia, and/or subjects with depression.

In one embodiment, the invention relates to a method for determining if a subject has schizophrenia using a Disease Vulnerability Index (DVI) to characterize the relationship between P_{[AVP]} and P_{Osm} during hypertonic solution infusion. The DVI can take the form of a linear discrimination function ƒ=c₁b+c₂t, where b is the estimated slope, t is the estimated threshold for an individual, and c₁ and c₂ are parameters to be estimated based on logistic regression with group (schizophrenia vs. normal control) as the dependent variable and ƒ as the linear predictor. Once c₁ and c₂ are estimated, a DVI score can be calculated for each individual. Different cutoffs then can be considered so that subjects with DVI scores higher than the cutoff can be predicted to be healthy, and subjects with DVI scores lower than the cutoff can be predicted to be schizophrenic. Thus, every possible cutoff value defines a diagnostic test, and since the status of each individual (healthy or schizophrenic) is known, the sensitivity and specificity of each test can be assessed. Based on these results, an ROC (Receiver Operating Characteristics) curve of sensitivity vs. one-minus specificity can be created based on different possible cutoffs of the DVI. A cutoff can then be selected that corresponds to the highest sensitivity and specificity for the sample such that sensitivity does not fall below 80%.

The DVI values can be calculated for each subject with depression according to the formula above described for subjects with schizophrenia. The DVI can also take the form of a non-linear discrimination function, therefore it is not restricted to a linear discriminant function.

As described herein, the diagnostic parameter of the present invention can be a parameter other than DVI, for example only the slope or threshold values based on the data. In one embodiment of the invention, a value of less than 0.060 for the slope of the regression line of P_{[AVP]} vs P_{Osm} is used to distinguish schizophrenic subjects from healthy subjects or subjects with depression, where a value of greater than 0.130 indicates a subject has vulnerability for depression. In one embodiment, a value of less than 0.040 for the slope of the regression line of P_{[AVP]} vs P_{Osm} is used to distinguish schizophrenic subjects from healthy subjects or subjects with depression. In various embodiments, the cutoff or reference value of the slope of the regression line of P_{[AVP]} vs P_{Osm} that is used in the method of the present invention for diagnosing schizophrenia can be any value in the range of 0 to 0.065, for example, 0.010, 0.020, 0.030, 0.050, or 0.062. In various embodiments, the cutoff or reference value of the slope of the regression line of P_{[AVP]} vs P_{Osm} that is used in the method of the present invention for diagnosing depression can be any value greater than 0.065, for example, 0.070, 0.080, 0.090, 0.100, 0.110, 0.120, or higher.

In one embodiment, the sensitivity of the DVI determination is greater than or equal to 80%. In one embodiment, a value of the slope of the regression line of P_{[AVP]} vs P_{Osm} for a subject is compared to the value of the slope of the regression line of P_{[AVP]} vs P_{Osm} for a healthy subject, depressed subject, or both, where a value less than that of the healthy or depressed subject indicates that the subject has vulnerability for schizophrenia. In one embodiment, a subject is diagnosed with schizophrenia when the value of the slope of the regression line of a plot of biomarker concentration versus blood plasma osmolality for the subject is less than a calculated cutoff based on the variability in a control population.

In various embodiments, the invention relates to a kit that can be used for the detection or diagnosis of schizophrenia and depression. In one embodiment, the kit comprises at least one biomarker assay, such as an assay for AVP, copeptin, or neurophysin II, a hypertonic solution for infusion, such as hypertonic saline solution, and instructional material. In one embodiment, the kit may also comprise materials, such as test tubes that can be used to retrieve biological samples from a subject. In one embodiment, the instructional material can be a publication, a recording, a diagram, or any other medium of expression, such as DVD, which can be used to communicate a method for detecting or diagnosing schizophrenia, such as calculation of the DVI for each individual. In one embodiment, the kit may also comprise an assay for measuring blood plasma osmolality.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, practice the claimed methods of the present invention. The following working examples therefore, specifically point out the preferred embodiments of the present invention, and are not to be construed as limiting in any way the remainder of the disclosure.

### Example 1

### Plasma Osmolality and AVP Concentration During Hypertonic Saline Administration

### Summary of Experimental Methods

A hypertonic (3.0% NaCl) saline infusion (HSI) was used to examine the osmotic regulation of AVP in the subjects. Hypertonic saline was infused at a rate of 0.1 ml·kg-1·min-1 for 120 minutes, which is designed to increase P_{Osm}, at about 15 mOsm/kg H₂O over this period. Blood is sampled at 15, 25, 35, 45, 60, 75, 90, 105, and 120 min during the infusion.

After the infusion, following a 30 min stabilization period, the subjects drank 15 ml/kg of plain water in 30 min. Following water ingestion, the subjects rested quietly for 60 min. Plasma osmolality and concentrations of AVP were determined in all blood samples.

P_{[AVP]} was determined after extraction from plasma as described previously (Freund B J, Claybaugh J R, Dice M S, Hashiro G M. Hormonal and vascular fluid responses to maximal exercise in trained and untrained males. J Appl Physiol. August 1987; 63(2):669-675. (PMID: 2958440)) on octadecylsilane cartridges (SEP-PAK C18, Waters Associates). This assay was highly specific for AVP with the antiserum prepared against a lysine vasopressin-thyroglobin conjugate and has a sensitivity of 0.6 pg/ml. P_{Osm}, was measured by freezing point depression (Advanced Instruments 3DII).

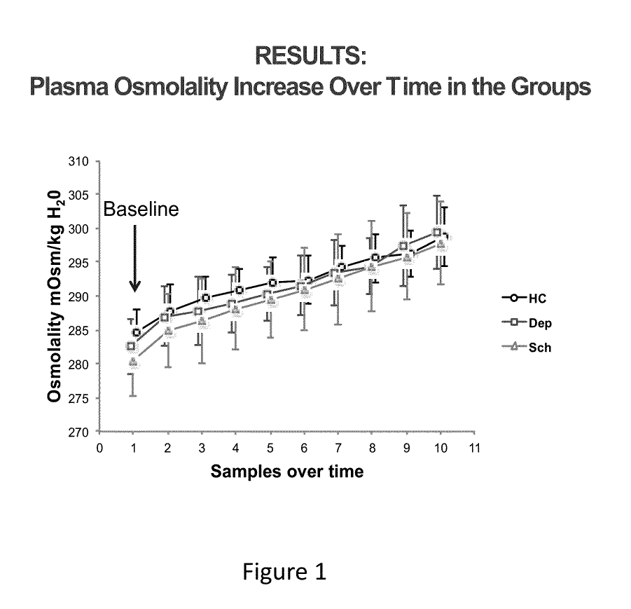

The P_{[AVP]} response to hypertonic saline was examined in healthy controls (N=5, all males; 1 Hispanic, 1 Asian and 1 Caucasian, and 2 African American; mean age: 28), schizophrenics (Sch; N=6; 5 males, 1 female; 3 African American, 3 Caucasian; mean age: 45; all treated with antipsychotics) and depressed subjects (Dep, N=6; 3 male and 3 female; 5 Caucasian, 1 Hispanic; mean age: 43; all but one treated with antidepressants) to assess the feasibility the approach described herein. None of the subjects had a history of polydipsia/hyponatremia. Hypertonic saline was administered (3.0% NaCl) at a rate of 0.1 ml·kg-1·min-1 for up to 2 h. Blood samples were collected at 15, 25, 35, 45, 60, 75, 90, 105, and 120 min. All subjects tolerated the procedures well. FIG. 1 shows the group mean P_{[AVP]} as a function of mean P_{Osm} during the hypertonic saline infusion (HSI), and Table 1 provides data on the individual responses.

It was found that all subjects showed a linear increase in P_{Osm} over time. The P_{Osm} increased by an average of 14 mOsmol/kgH₂O in the healthy control, 16 mOsmol/kgH₂O in the depressed, and 17 mOsmol/kgH₂O in the schizophrenia group. As demonstrated herein, P_{[AVP]} response to P_{Osm} in subjects with schizophrenia was lower than in healthy controls and subjects with depression. In fact, despite an increase of 17 mOsmol/kg H₂O, P_{[AVP]} did not appreciably increase in this group. It was also found that the depressed group showed higher P_{[AVP]} response to P_{Osm} than the healthy control group.

In Table 1, the individual values of the slopes of the regression lines of P_{[AVP]} response to P_{Osm} are shown. The two schizophrenic subjects showed the lowest two values, and the highest values belonged to the depressed subjects.

TABLE 1
Slope and Threshold Values of P_{[AVP]} Response
to P_{Osm} during HSI in individual subjects

| | HC1 | HC2 | HC3 | HC4 | HC5 |
|---|---|---|---|---|---|
| Slopes | 0.066 | 0.068 | 0.061 | 0.126 | 0.063 |
| Thresholds | 272 | 272 | 269 | 279 | 268 |

| | Dep1 | Dep2 | Dep3 | Dep4 | Dep5 | Dep6 |
|---|---|---|---|---|---|---|
| Slopes | 0.166 | 0.068 | 0.345 | 0.111 | 0.061 | 0.296 |
| Thresholds | 276 | 269 | 282 | 286 | 268 | 274 |
| | Sch1 | Sch2 | Sch3 | Sch4 | Sch5 | Sch6 |
| Slopes | 0.008 | 0.004 | 0.024 | 0.034 | 0.044 | 0.057 |
| Thresholds | 141 | 63 | 199 | 243 | 260 | 251 |

HC: Healthy control,
Dep: Depressed subject,
Sch: Schizophrenic subject

These data support the finding that the P_{[AVP]} response to P_{Osm} in schizophrenic subjects is remarkably distinguishable from the other two groups, especially from the depressed group. These findings also highlight the fact that even in schizophrenic subjects without a history of polydipsia/hyponatremia, underlying AVP deficits can be revealed using the HSI paradigm. Collectively, these findings suggest that the approach described herein may yield a viable a biomarker for schizophrenia.

1. A method of diagnosing schizophrenia in a subject, comprising:
administering a hypertonic solution to a subject;
measuring blood plasma osmolality in the subject;
measuring the concentration of at least one biomarker in the subject;
determining the value of a diagnostic parameter for the subject using the blood plasma osmolality in the subject and the concentration of at least one biomarker in the subject; and
diagnosing the subject with schizophrenia when the value of the diagnostic parameter for the subject is less than a reference value of the diagnostic parameter.

2. The method of claim 1, wherein the diagnostic parameter is a Disease Vulnerability Index (DVI), based on a calculation of the slope and threshold values derived from the regression line of a plot of biomarker concentration versus blood plasma osmolality.

3. The method of claim 1, wherein the diagnostic parameter is the value of the slope of the regression line of a plot of biomarker concentration versus blood plasma osmolality.

4. The method of claim 3, wherein the reference value for the diagnostic parameter is about 0.060.

5. The method of claim 1, wherein the diagnostic parameter is the threshold value of a plot of biomarker concentration versus blood plasma osmolality.

6. The method of claim 5, wherein the reference value for the diagnostic parameter is about 260.

7. The method of claim 1, wherein the biomarker is arginine-vasopressin.

8. The method of claim 1, wherein the biomarker is neurophysin II.

9. The method of claim 1, wherein the biomarker is copeptin.

10. The method of claim 1, wherein the reference value of the diagnostic parameter is determined by measuring the blood plasma osmolality and the concentration of at least one biomarker in a healthy control subject.

11. A method of diagnosing depression in a subject, comprising:
administering a hypertonic solution to a subject;
measuring blood plasma osmolality in the subject;
measuring the concentration of at least one biomarker in the subject;
determining the value of a diagnostic parameter for the subject using the blood plasma osmolality in the subject and the concentration of at least one biomarker in the subject; and
diagnosing the subject with depression when the value of the diagnostic parameter for the subject is greater than a reference value of the diagnostic parameter.

12. The method of claim 11, wherein the diagnostic parameter is a Disease Vulnerability Index (DVI), based on a calculation of the slope and threshold values derived from the regression line of a plot of biomarker concentration versus blood plasma osmolality.

13. The method of claim 11, wherein the diagnostic parameter is the value of the slope of the regression line of a plot of biomarker concentration versus blood plasma osmolality.

14. The method of claim 13, wherein the reference value for the diagnostic parameter is about 0.130.

15. The method of claim 11, wherein the biomarker is arginine-vasopressin.

16. The method of claim 11, wherein the biomarker is neurophysin II.

17. The method of claim 11, wherein the biomarker is copeptin.

18. The method of claim 11, wherein the reference value of the diagnostic parameter is determined by measuring the blood plasma osmolality and the concentration of at least one biomarker in a healthy control subject.

19. A kit for detecting or diagnosing schizophrenia or depression, comprising at least one biomarker assay, a hypertonic solution for infusion, and instructional material.

20. The kit of claim 19 wherein the biomarker assay is for measuring arginine-vasopressin concentration.

21. The kit of claim 19, wherein the biomarker assay is for measuring copeptin concentration.

22. The kit of claim 19, wherein the biomarker assay is for measuring neurophysin II concentration.

23. The kit of claim 19, further comprising an assay for measuring blood plasma osmolality.

24. The kit of claim 19, wherein the assay is based on a monoclonal antibody.

25. The kit of claim 19, wherein the assay is based on a polyclonal antibody.
